(19) United States
(12) Patent Application Publication
(10) Pub. No.: US 2024/0393351 A1
(43) Pub. Date: Nov. 28, 2024

(54) HEMOGLOBIN ADVANCED GLYCATION END PRODUCTS MEASURING INSTRUMENT AND MEASURING CONTAINER THEREOF

(71) Applicant: PolyVisions Technology Co., Ltd., NEW TAIPEI CITY (TW)

(72) Inventor: Chih-Ju LIN, NEW TAIPEI CITY (TW)

(73) Assignee: PolyVisions Technology Co., Ltd., NEW TAIPEI CITY (TW)

(21) Appl. No.: 18/428,326

(22) Filed: Jan. 31, 2024

(30) Foreign Application Priority Data
May 23, 2023 (CN) 202310581376.5

(51) Int. Cl. G01N 33/72; B01L 3/00

(52) U.S. Cl. CPC G01N 33/726; B01L 3/502715; G01N 33/723; B01L 2200/025; B01L 2200/04; B01L 2200/16; B01L 2300/0654; B01L 2300/168

(57) ABSTRACT

A hemoglobin advanced glycation end products measuring instrument includes a measuring base, a measuring container, a light emitting unit, a first photosensitive assembly, a second photosensitive assembly, and a processor. The light emitting unit outputs parallel light beam when being driven. The parallel light beam irradiates to-be-tested liquid in the measuring container to generate fluorescence and a transmission light beam. The first photosensitive assembly is positioned at the first optical axis, and receiving and converting the transmission light beam with a first preset wavelength into first light intensity. The second photosensitive assembly is positioned at the second optical axis, and receiving and converting the fluorescence with a second preset wavelength into a second light intensity. The processor obtains a hemoglobin advanced glycation end products measuring result according to the first light intensity, the second light intensity, the first standard intensity and the first empty intensity.

### CROSS-REFERENCE TO RELATED APPLICATION

This non-provisional application claims priority under 35 U.S.C. § 119 (a) to patent application No. 202310581376.5 filed in China on May 23, 2023, the entire contents of which are hereby incorporated by reference.

### BACKGROUND

### Technical Field

The present application belongs to the field of diabetes measuring, and particularly relates to a hemoglobin advanced glycation end products measuring instrument and a measuring container thereof.

### Related Art

At present, for the measuring and control of diabetes symptoms, the main method is to measure the glycated hemoglobin (glycohemoglobin, or HbAlc) of the blood to track the blood glucose status of patients. The HbAlc value is purported to the glycated hemoglobin by glucose, which mainly reflects the weighted average of plasma glucose over a period of time.

The important impact of glycated hemoglobin is to easily cause a Maillard reaction in a high-concentration glucose environment. In the blood, the carbonyl group of glucose will react with the amino group of arginine or lysine on proteins, nucleic acids or fats accumulated produce advanced glycation end products (AGEs). The accumulation of advanced glycation end products is the real result of tissue glycation. The production of advanced glycation end products is systemic, it will be presented in blood, tissues, and almost all organs. It can induce inflammatory responses easily, which can lead to cardiovascular diseases, renal diseases, cataracts, or peripheral neuropathy. They are currently treated as the main causes of diabetic complications. Therefore, the accumulation of advanced glycation end products can help to understand the glycated rates of organs and tissues, further to understand the lesion rates of organs and tissues, finally it will be a good tool of tracking long-term glycation index.

However, glucometers currently sold on the market are configured to measure the glucose concentration in plasma. More advanced, clinic tests are generally to measure the glycated hemoglobin in hospital, but rarely to measure the advanced glycation end products. In clinical practice, Enzyme-linked immunosorbent assay (ELISA) is adopted to measure the advanced glycation end products, which can accurately measure the advanced glycation end products in blood and tissues. However, the ELISA test is cumbersome, highly technical and time-consuming, and it is not suitable for the general public to conduct self-testing at home.

### SUMMARY

In view of this, according to some embodiments, a hemoglobin advanced glycation end products measuring instrument is provided and includes a measuring base, a measuring container (microcuvette), a light emitting unit, a first photosensitive assembly, a second photosensitive assembly, and a processor. The measuring base includes a main body, a first optical axis and a second optical axis. The main body is provided with an accommodated space. The second optical axis and the first optical axis are intersected in the accommodated space by an included angle. The measuring container accommodates to-be-tested liquid. The measuring container is positioned in the accommodated space to enable the to-be-tested liquid to correspond to the intersection of the first optical axis and the second optical axis. The light emitting unit is positioned at one end of the first optical axis of the measuring base, and outputs parallel light beam along the first optical axis when being driven. The parallel light beam irradiates the to-be-tested liquid to generate fluorescence, and a transmission light beam is towards the other end of the first optical axis. The first photosensitive assembly is positioned at the other end of the first optical axis of the measuring base, and receives and converts the transmission light beam with a first preset wavelength into a first light intensity. The second photosensitive assembly is positioned at one end of the second optical axis, and receives and converts the fluorescence with a second preset wavelength into a second light intensity. The processor obtains a hemoglobin advanced glycation end products measuring result according to the first light intensity, the second light intensity, a first standard intensity and a first empty intensity.

According to some embodiments, the light emitting unit includes a light emitting diode and a first lens group. The light emitting diode emits a light source when being driven. The first lens group receives the light source and outputs the parallel light beam.

According to some embodiments, the first lens group includes a first lens, a second lens and a third lens. The first lens receives the light source and outputs a convergent light beam. The second lens receives the convergent light beam and outputs a focused light beam. The third lens receives the focused light beam and outputs the parallel light beam.

According to some embodiments, the first preset wavelength is between 330 and 390 nanometers (nm), the second preset wavelength is between 420 and 520 nm.

According to some embodiments, the included angle is between 45-135 degrees.

According to some embodiments, the processor obtains the hemoglobin advanced glycation end products measuring result is according to the following equation: M₁=M₀*(N₀−N₁)/(N₀−N). M₁ represents the hemoglobin advanced glycation end products measuring result, M₀ represents the second light intensity, No represents the first empty intensity, N₁ represents the first standard intensity, and N represents the first light intensity.

According to some embodiments, the first photosensitive assembly includes a first optical filter, a fourth lens and a first photosensitive element which are sequentially arranged from the measuring container to the other end of the first optical axis. The first optical filter enables the transmission light beam with the wavelength between 330 and 390 nm to transmit through. The fourth lens focuses the transmission light beam which transmits through the first optical filter on the first photosensitive element. The first photosensitive element receives and converts the transmission light beam focused by the fourth lens into the first light intensity.

According to some embodiments, the second photosensitive assembly includes a fifth lens, a second optical filter, a sixth lens and a second photosensitive element which are sequentially arranged from the measuring container to one end of the second optical axis. The fifth lens receives the fluorescence and outputs a parallel beam of fluorescence; the second optical filter enables the parallel beam of fluorescence with the wavelength of 420 nm to 520 nm to transmit through; the sixth lens focuses the parallel beam of fluorescence which transmit through the second optical filter on the second photosensitive element; and the second photosensitive element receives and converts the focused fluorescence into the second light intensity.

According to some embodiments, the measuring container includes a liquid inlet section and a measuring section. The liquid inlet section is provided with a channel and an opening communicating with the channel. The measuring section is connected to the liquid inlet section and is provided with a measuring tank. The measuring tank communicates with the channel and is configured to accommodate to-be-tested liquid. The measuring tank is provided with a first main light-penetrating part, a second main light-penetrating part and a side light-penetrating part. The first main light-penetrating part and the second main light-penetrating part correspond to the first optical axis, and the side light-penetrating part corresponds to the second optical axis.

According to some embodiments, the first main light-penetrating part and the second main light-penetrating part enable the parallel light beam to transmit through. The side light-penetrating part enables the fluorescence to transmit through.

According to some embodiments, the inner surface of the measuring tank and the inner surface of the channel are smooth surfaces.

The detailed features and advantages of the present application are described in detail below in the embodiments, and their content is sufficient to enable any person of ordinary skill in the art to understand the technical content of the present application and implement it accordingly. According to the content disclosed in this specification, the claims and the accompanying drawings, any person of ordinary skill in the art can easily understand the relevant purposes and advantages of the present application.

### BRIEF DESCRIPTION OF THE DRAWINGS

FIG. 1A is a three-dimensional schematic diagram of a hemoglobin advanced glycation end products measuring instrument according to some embodiments.

FIG. 1B is a structural schematic diagram of a hemoglobin advanced glycation end products measuring instrument according to some embodiments.

FIG. 2 is a structural schematic diagram of a hemoglobin advanced glycation end products measuring instrument according to some embodiments.

FIG. 3 is a three-dimensional schematic diagram of a measuring container according to some embodiments.

FIG. 4 is a top view of a measuring container in FIG. 3.

FIG. 5 is a structural schematic diagram of a hemoglobin advanced glycation end products measuring instrument according to some embodiments, and shows a structure of the measuring instrument without a measuring container.

### DETAILED DESCRIPTION

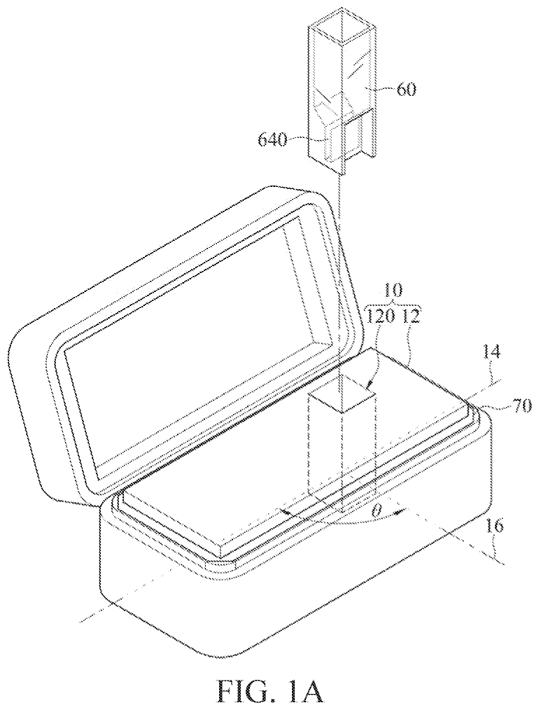

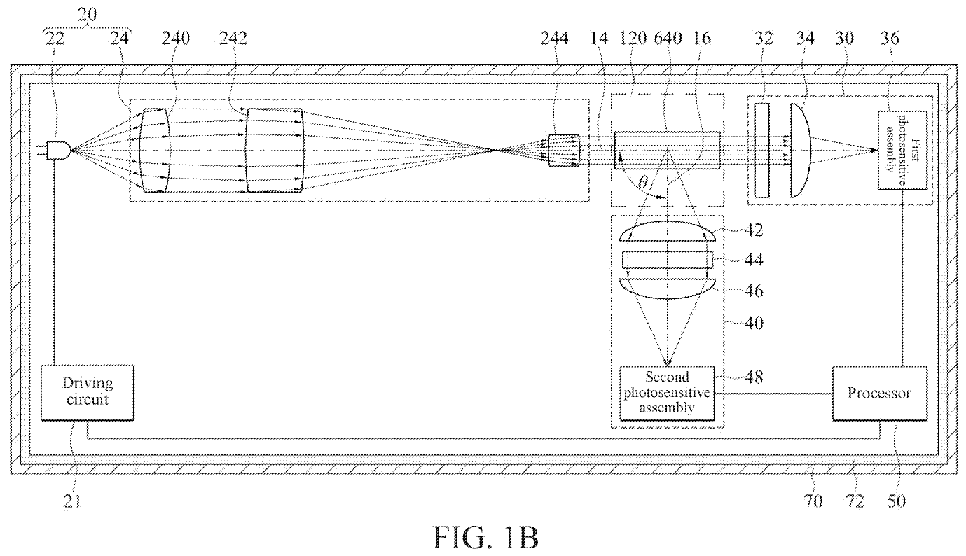

As shown in FIG. 1A and FIG. 1B, FIG. 1A is a three-dimensional schematic diagram of a hemoglobin advanced glycation end products measuring instrument according to some embodiments, and FIG. 1B is a structural schematic diagram of a hemoglobin advanced glycation end products measuring instrument according to some embodiments. The hemoglobin advanced glycation end products measuring instrument includes a measuring base 10, a measuring container 60, a light emitting unit 20, a first photosensitive assembly 30, a second photosensitive assembly 40 and a processor 50.

As shown in FIG. 1A and FIG. 1B, the measuring base 10 includes a main body 12, a first optical axis 14 and a second optical axis 16. The main body 12 is provided with an accommodated space 120. The second optical axis 16 and the first optical axis 14 are intersected in the accommodated 120 space by an included angle θ. The range of the included angle θ is not limited as long as the second photosensitive assembly 40 can receive fluorescence (detailed later). In some embodiments, the first optical axis 14 and the second optical axis 16 are phantom lines to indicate the light advancing route. In some embodiments, the included angle θ is 45-135 degrees. In some embodiments, the included angle θ is 90 degrees.

As shown in FIG. 1A, the measuring container 60 is configured to accommodate to-be-tested liquid, and the measuring container 60 is positioned in the accommodated space 120 to enable the to-be-tested liquid to corresponds to the intersection of the first optical axis 14 and the second optical axis 16. Specifically, in some embodiments, the to-be-tested liquid is positioned in a measuring tank 640 of the measuring container 60, the measuring tank 640 corresponds to the intersection of the first optical axis 14 and the second optical axis 16 (as shown in FIG. 1B), and the structure of the measuring container 60 is detailed later. In some embodiments, the to-be-tested liquid includes blood and a reaction solution. The blood is taken from a to-be-tested person. In some embodiments, the reaction solution includes a mixed solution of Ethylenediaminetetraacetic acid (EDTA), Tris(hydroxymethyl)aminomethane (Tris) and Phenylmethanesulfonyl fluoride (PMSF). The reaction solution may react with the blood to rupture membranes of red blood cells, so that light beam with a first preset wavelength may excite the fluorescent and emit fluorescence with a second preset wavelength after irradiating the to-be-tested liquid. In some embodiments, the volume of the reaction solution is 0.2 mL (mini liter), the concentration of the EDTA is 0.1-200 mM (mini molarity), the concentration of the Tris is 1 mM-1 M, the concentration of the PMSF is 0.1-10 mM, and a drop of blood is extracted (about 0.03 mL).

As shown in FIG. 1B, the light emitting unit 20 is positioned at one end of the first optical axis 14 of the measuring base 10 (left in FIG. 1B), and outputs parallel light beam along the first optical axis 14 when being driven. The parallel light beam irradiates the to-be-tested liquid to generate fluorescence and transmission light beam towards the other end of the first optical axis 14 (right in FIG. 1B). The transmission light beam will travel along the first optical axis 14 to be received by the first photosensitive assembly 40, and the fluorescence travels along the second optical axis 16 to be received by the second photosensitive assembly 40. In some embodiments, the measuring base 10, the measuring container 60, the light emitting unit 20, the first photosensitive assembly 30 and the second photosensitive assembly 40 are arranged in a chamber 70 (in the main body 12 shown as FIG. 1A), and in addition, the inner side wall of the chamber 70 is provided with a light absorption layer 72 for absorbing excessive photons, so that the interference on the measuring due to reflection of the radiated light in the chamber 70 can be avoided. In addition, in some embodiments, the light absorption layer 72 is formed by coating black paint onto the inner side wall of the chamber 70. In some embodiments, the diameter of the parallel light beam is about 1 mm.

As shown in FIG. 1B, the first photosensitive assembly 30 is positioned at the other end of the first optical axis 14 of the measuring base 10 (right in FIG. 1B). That is, the light emitting unit 20 is positioned at the initial end of the first optical axis 14, and the first photosensitive assembly 30 is positioned at the tail end of the first optical axis 14. The first photosensitive assembly 30 receives and converts the transmission light beam with the first preset wavelength into the first light intensity. In some embodiments, the first preset wavelength is a wavelength range between 330 nm and 390 nm.

As shown in FIG. 1B, the second photosensitive assembly 40 is positioned at one end of the second optical axis 16 (lower side in FIG. 1B). The initial end of the second optical axis 16 is positioned in the measuring container 60 while the tail end is positioned in the second photosensitive assembly 40. The second photosensitive assembly 40 receives and converts the fluorescence with the second preset wavelength into the second light intensity. In some embodiments, the second preset wavelength is a wavelength range between 420 nm and 520 nm. As mentioned above, in some embodiments, an included angle θ is 45-135 degrees, the included angle θ is determined based on whether the second photosensitive assembly 40 can receive and convert the fluorescence with the second preset wavelength into the second light intensity, which is on the basis of the fluorescence, so the fluorescence does not have a specific main optical axis; and therefore, the range of the included angle θ can be 45-135 degrees, and the included angle θ can even be in a larger range as long as the second photosensitive assembly 40 can receive and convert fluorescence with the second preset wavelength into the second light intensity.

The determination of the first preset wavelength and the second preset wavelength are shown in the following table. It can be seen from the following table that advanced glycation end products fluorescent molecules may include pentosidine, vesperlysineslike, vesperlysine A, vesperlysine B, vesperlysine C, lysyl-pyrropyridine, (FFI, 2-(2-furoyl)-4 (5)-(2-furanyl)-1H-imidazole), Argpyrimidine, Crossline, and Fluorolink. An Excitation (Absorption) peak refers to the peak wavelength of the light beam absorbed by the advanced glycation end products molecules. The electrons of fluorescent molecules transit from the ground state to an excited state after absorbing excitation photons. The electrons will emit fluorescent photons under the influence of molecular oscillation and back to the ground state. The peak of the wavelength of the fluorescent photons is an Emission peak of the advanced glycation end products. For example as shown on the table, the advanced glycation end products is a multi-molecule mixed substance, and the optimal excitation wavelength of pentosidine and Argpyrimidine is about 335 nm. When the pentosidine is irradiated at the excitation peak of 335 nm, the excited emission peak wavelength is 385 nm and usually the wavelength range (spectrum) of the emission fluorescence is from 382 to 395 nm. In other words, as long as the spectrum of the parallel light beam outputted by the light emitting unit 20 convers 335 nm, the pentosidine of the to-be-tested liquid is irradiated to emit fluorescence with 382-395 nm. Likewise, when the vesperlysine A is irradiated at the Excitation peak of 366 nm, the excited emission peak wavelength is 442 nm.

| Advanced glycation end | Excitation | Emission |
|---|---|---|
| products molecules | peak (nm) | peak (nm) |
| pentosidine | 335 | 385 |
| vesperlysineslike | 370 | 440 |
| vesperlysine A | 366 | 442 |
| vesperlysine B | 366 | 442 |
| vesperlysine C | 345 | 405 |
| lysyl-pyrropyridine | 370 | 448 |
| FFI | 380 | 440 |
| Argpyrimidine | 335 | 400 |
| Crossline | 379 | 463 |
| Fluorolink | 380 | 460 |

In some embodiments, the spectrum of the parallel light beam outputted by the light emitting unit 20 is from 335 to 385 nm. Accordingly, the parallel light beam could irradiate the substance of the advanced glycation end products molecules shown on the above table and the advanced glycation end products molecules will emit fluorescence. The fluorescence emitted from the advanced glycation end products molecules will include fluorescence with the Emission peaks' wavelengths (385 to 460 nm) and fluorescence with all spectral widths. In order for the second photosensitive assembly 40 to receive and convert most of the fluorescence in the embodiments, the second preset wavelength is a wavelength range between 385 to 520.

The first preset wavelength may relate to the spectrum of the parallel light beam outputted from the light emitting unit 20. In some embodiments, the spectrum of the parallel light beam covers some of optimal excitation wavelengths of the advanced glycation end products. As shown in the above table, if the spectrum of the parallel light beam covers 335 to 385 nm and the substance of the advanced glycation end products molecules shown on the above table will be excited to emit fluorescence. Thus, the first preset wavelength may be from 335 to 385 nm. In some embodiments, the first preset wavelength is from 330 nm to 390 nm in consideration of the spectral width of the parallel light beam outputted by the light emitting unit 20. However, it is not necessary for the parallel light beam to cover all of the optimal excitation peaks. In some embodiments, the spectrum of the parallel light beam is from 360 to 380 nm and the first preset wavelength may be from 360 to 380 nm or from 330 to 390 nm.

In some embodiments, in order to make sure that the first photosensitive assembly 30 receives the transmission light beam with the first preset wavelength and the second photosensitive assembly 40 receives the fluorescence with the second preset wavelength, the first preset wavelength and the second preset wavelength have no overlap. In some embodiments, the upper limit of the first preset wavelength is 20 nm or 30 nm smaller than the lower limit of the second preset wavelength. For example, the first preset wavelength is from 330 to 390 nm and the second preset wavelength is from 410 to 520 nm. In some embodiments, the first preset wavelength is from 330 to 390 nm and the second preset wavelength is from 420 to 520 nm.

As shown in FIG. 1B, the processor 50 obtains the hemoglobin advanced glycation end products measuring results according to the first light intensity, the second light intensity, the first standard intensity and the first empty intensity. In some embodiments, the hemoglobin advanced glycation end products measuring result is a measuring value, and the processor 50 obtains the measuring value of the hemoglobin advanced glycation end products measuring result according to the equation:

$${M}_{1} = {M}_{0} * \frac{{N}_{0} - {N}_{1}}{{N}_{0} - N} .$$

M₁ represents the measuring value; N represents the first light intensity, which refers to the number of photons with the first preset wavelength measured by the first photosensitive assembly 30 after the measuring container 60 filled with the to-be-tested liquid is positioned in the accommodated space 120, and the light emission by the light emitting unit 20 transmits through the to-be-tested liquid; M₀ represents the second light intensity, which refers to the number of photons with the second preset wavelength measured by the second photosensitive assembly 40 after the measuring container 60 filled with the to-be-tested liquid is positioned in the accommodated space 120, and the light emission by the light emitting unit 20 transmits through the to-be-tested liquid; N₁ represents the first standard intensity, which refers to the number of photons with the first preset wavelength measured by the first photosensitive assembly 30 after the measuring container 60 filled with the reaction solution is positioned in the accommodated space 120, and the light emission by the light emitting unit 20 transmits through the reaction solution; and No represents the first empty intensity, which refers to the number of photons with the second preset wavelength measured by the first photosensitive assembly 30 after the measuring container 60 is positioned in the accommodated space 120 and the measuring container 60 is free of any liquid, and the light emission by the light emitting unit 20 transmits through the measuring container 60

$$\frac{{N}_{0} - {N}_{1}}{{N}_{0} - N}$$

is adopted for concentration correction for the difference between the volume of blood dripped each time and the hemoglobin concentration of each sample, so N₁ is possibly greater than or smaller than N.

In some embodiments, the hemoglobin advanced glycation end products measuring result is a glycation rate level code (also called a glycation rate code); the glycation rate level code is used for determining the glycation rate according to the fluorescence value, and the code can be specified according to the requirements of a user. For example, as shown in the following table, the hemoglobin advanced glycation end products measuring instrument in any one of the embodiments mentioned above takes advanced glycation end products liquid, generated after standard hemoglobin is subjected to glucose water culture, as standard liquid (namely the mentioned above to-be-tested liquid); the hemoglobin concentration is 0.35 mM (Optical density, O.D.) about 0.5); and the fluorescence intensity is measured by different grades of standard liquids, so as to establish corresponding codes in advance. For example, the liquid with glucose concentration of 5 mM, 7.5 mM, 10 mM, 20 mM and 30 mM is correspondingly used as the standard liquid, and the corresponding equivalent blood glucose values (mg/dL, mg/deciliter) is correspondingly 90,135,180, 360, and 540. As the average life of red blood cells is about 90 days, the average age of the whole red blood cells is 45 days, the 45-day standard is adopted for in-vitro culture of hemoglobin glycation, and the ambient temperature is continuously 37° C. Therefore, the measuring value measured after the parallel light beam transmits through the to-be-tested liquid with different glucose concentrations is used as a reference for determining the glycation rate (namely the mentioned above glycation rate level code). In this example, the glycation rate level codes are A to E, A represents that the glycation rate is low, B represents that the glycation rate is standard, C represents that the glycation rate is slightly high, D represents that the glycation rate is high, and E represents that the glycation rate is higher, and the condition is serious.

| Glucose | Equivalent blood glucose | Hemoglobin glycation |
|---|---|---|
| concentration (mM) | value(mg/dL) | fluorescence |
| 5.0 | 90 | A |
| 7.5 | 135 | B |
| 10 | 180 | C |
| 20 | 360 | D |
| 30 | 540 | E |

When the hemoglobin advanced glycation end products measuring instrument is used, a drop of blood is taken from the body of the patient (such as a finger) with a blood taking needle; the blood is dripped into a reaction solution in the measuring container 60 to form the to-be-tested liquid, and then is shaken or stirred, so that the blood sufficiently reacts with the reaction solution; and then, still standing is performed for several minutes to form the to-be-tested liquid. Then, the measuring container 60 is put into the accommodated space 120, the light emitting unit 20 is started, and thus the parallel light beam transmits through the to-be-tested liquid to generate the transmission light beam and fluorescence to be correspondingly received by the first photosensitive assembly 30 and the second photosensitive assembly 40. Then, the processor 50 obtains the hemoglobin advanced glycation end products measuring result according to the first light intensity, the second light intensity, the first standard intensity and the first empty intensity, and thus the patient can determine the glycation condition by their selves.

The advanced glycation end products (AGEs) are chemical products obtained by Maillard reaction of sugar molecules and protein. This phenomenon is common presented in diabetes and aging, so the advanced glycation end products generally exist in most organs of the body, and the more the molecules of the advanced glycation end products are carried by the organs along with the severity. Therefore, the glycation degree can be effectively determined by detecting the number of the advanced glycation end products. The advanced glycation end products are mixtures, and part of molecules in the advanced glycation end products can be emitted fluorescence via absorbing the photons, so the glycation degree can be effectively determined by using the characteristic that the advanced glycation end products fluorescence are fluorescents. In addition, the measuring of the advanced glycation end products by the mentioned above hemoglobin advanced glycation end products measuring instrument embodiment is performed on normal people and diabetic patients, the to-be-tested liquid of the normal people and the diabetic patients is excited by the light source with the peak wavelength of 370 nm, and the result shows that the fluorescence intensity measured from the to-be-tested liquid of the diabetic patients is higher than that measured from the normal people's. Particularly, when the wavelength is between 460 and 480 nm, the difference of the fluorescence intensity between the diabetic patients and the normal people is more obvious. Therefore, the diabetes can be effectively determined by using the hemoglobin advanced glycation end products measuring instrument to measure the advanced glycation end products.

As shown in FIG. 1B, in some embodiments, the light emitting unit 20 includes a light emitting diode 22 and a first lens group 24. The light emitting diode 22 emits the light source when being driven, and the first lens group 24 receives the light sources and then outputs the parallel light beam. In some embodiments, the light emitting diode 22 can emit a light source with the wavelength between about 330 nm and about 390 nm, and the power of the light emitting diode 22 is about 80 mW. In some embodiments, the light emitting unit 20 includes a driving circuit 21, the driving circuit 21 is connected to the processor 50 and the light emitting diode 22, and the driving circuit 21 is controlled by the processor 50 to drive the light emitting diode 22 to emit the mentioned above light source.

As shown in FIG. 1B, in some embodiments, the first lens group 24 includes a first lens 240, a second lens 242 and a third lens 244. The first lens 240 receives the light source and outputs a convergent light beam. The second lens 242 receives the convergent light beam and outputs a focused light beam. The third lens 244 receives the focused light beam and outputs parallel light beam.

As shown in FIG. 1B, in some embodiments, the first photosensitive assembly 30 includes a first optical filter 32, a fourth lens 34 and a first photosensitive element 36 which are sequentially arranged from the measuring container 60 to the other end of the first optical axis 14 (sequentially arranged from left to right as shown in FIG. 1B); the first optical filter 32 enables the transmission light beam with the wavelength between 330 and 390 nm to transmit through; the fourth lens 34 focuses the transmission light beam which transmits through the first optical filter 32 on the first photosensitive element 36; and the first photosensitive element 36 receives and converts the transmission light focused by the fourth lens 34 into the first light intensity. In some embodiments, the fourth lens 34 focuses the transmission light beam at the center of a receiving surface of the first photosensitive element 36.

As shown in FIG. 1A and FIG. 1B, in some embodiments, the second photosensitive assembly 40 includes a fifth lens 42, a second optical filter 44, a sixth lens 46 and a second photosensitive element 48 which are sequentially arranged from the measuring container 60 (specifically, the measuring tank 640 of the measuring container 60, or the accommodated space of the measuring container 60) to one end of the second optical axis 16 (sequentially arranged from top to bottom as shown in FIG. 1B). The fifth lens 42 receives the fluorescence and outputs the parallel light beam of fluorescence; the second optical filter 44 enables the parallel light beam of fluorescence with the wavelength between 420 and 520 nm to transmit through; the sixth lens 46 focuses the parallel light beam of fluorescence which transmits through the second optical filter 44 on the second photosensitive element 48; and the second photosensitive element 48 receives and converts the focused fluorescence into the second light intensity. In some embodiments, the optical density (O.D. value) of the second optical filter 44 is larger than 6, so that laser can be filtered out.

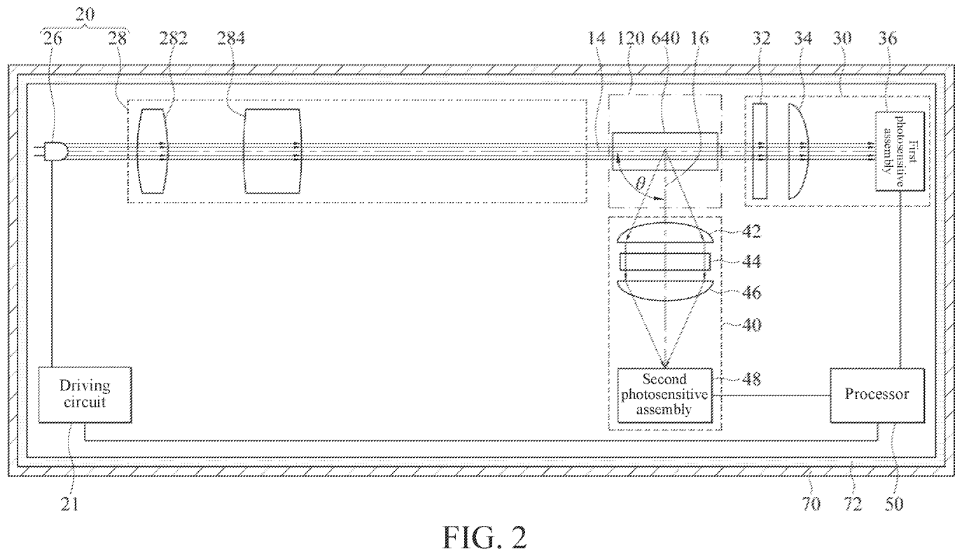

As shown in FIG. 1A and FIG. 2, in some embodiments, the light emitting unit 20 includes a laser assembly 26 and a beam reducer group 28. The laser assembly 26 emits laser when being driven. The beam reducer group 28 reduces the laser to output parallel light beam. The beam reducer group 28 reduces the optical path (beam diameter) of the laser to enable the laser to completely transmit through the to-be-tested liquid in the measuring container 60 (the to-be-tested liquid in the measuring tank 640). In some embodiments, the beam reducer group 28 includes a seventh lens 282 and an eighth lens 284. The seventh lens 282 is configured to focus and output the laser, and the eighth lens 284 is configured to output the focused laser in a parallel mode. The sectional area (optical path) of the laser entering the beam reducer group 28 is larger than the sectional area of the laser transmitting out of the beam reducer group 28.

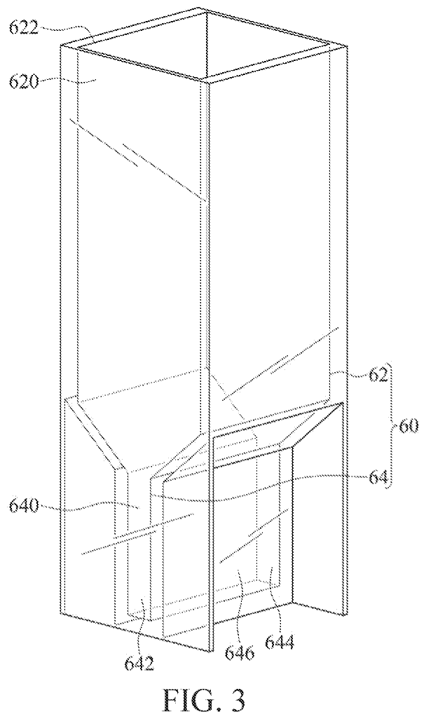

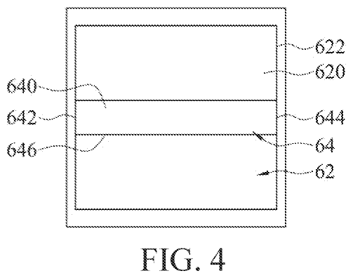

As shown in FIG. 3 and FIG. 4, in some embodiments, the measuring container 60 includes a liquid inlet section 62 and a measuring section 64. The liquid inlet section 62 is provided with a channel 620 and an opening 622. The channel 620 communicates with the opening 622. The measuring section 64 is connected to the liquid inlet section 62 and is provided with the measuring tank 640, and the measuring tank 640 communicates with the channel 620 and is configured to accommodate the to-be-tested liquid. The opening 622 is a liquid inlet, and blood and the reaction solution enter the measuring tank 640 through the opening 622 and the channel 620. The measuring tank 640 is provided with a first main light-penetrating part 642, a second main light-penetrating part 644 and a side light-penetrating part 646 (namely three adjacent tank walls corresponding to the measuring tank 640, or called side walls). The first main light-penetrating part 642 and the second main light-penetrating part 644 correspond to the first optical axis 14, and the side light-penetrating part 646 corresponds to the second optical axis 16, that is, when the measuring container 60 is placed in an accommodated space 120, the first optical axis 14 penetrates through the first main light-penetrating part 642 and the second main light-penetrating part 644. The side light-penetrating part 646 is positioned on the second optical axis 16, that is, when the measuring container 60 is placed in the accommodated space 120, the second optical axis 16 penetrates through the side light-penetrating part 646. The light-transmitting wavelength of the first main light-penetrating part 642 and the second main light-penetrating part 644 enable the light beam with the wavelength between 330 and 390 nm to transmit through, and the light-transmitting wavelength of the side light-penetrating part 646 enables the light beam with the wavelength between 420 and 520 nm to transmit through. But it is not limited to this, the light-transmitting wavelength of the first main light-penetrating part 642, the second main light-penetrating part 644 and the side light-penetrating part 646 may be within a larger range, for example, the light beam with the wavelength between 300 and 600 nm can transmit through. In some embodiments, the first main light-penetrating part 642 and the second main light-penetrating part 644 enable the parallel light beam to transmit through, and the side light-penetrating part 646 enables the fluorescence to transmit through. Therefore, after the parallel light beam transmits through the first main light-penetrating part 642 along the first optical axis 14, the to-be-tested liquid is excited to generate the fluorescence and the transmission light beam; and the transmission light beam transmits through the second main light-penetrating part 644 along the first optical axis 14 to be received by the first photosensitive assembly 30. The fluorescence transmits through the side light-penetrating part 646 along the second optical axis 16 to be received by the second photosensitive assembly 40.

In some embodiments, the first main light-penetrating part 642, the side light-penetrating part 646 and the second main light-penetrating part 644 correspond to three adjacent tank walls (or called side walls) of the measuring tank 640 and can be all or a part of the corresponding tank walls. For example, the first main light-penetrating part 642 corresponds to a part of the tank walls, and the size of the part is larger than or equal to the optical path of parallel light beam, so that the parallel light beam can enter the measuring tank 640 and excite the to-be-tested liquid. Similarly, the second main light-penetrating part 644 corresponds to a part of the tank walls, and the size of the part is larger than or equal to the optical path of the parallel light beam, so that the parallel light beam can enter the measuring tank 640 and excite the to-be-tested liquid. The side light-penetrating part 646 corresponds to a part of the tank walls, and the part may occupy most of the area of the corresponding tank walls, which facilitates the emitted fluorescence to transmit out.

In some embodiments, the inner surface of the measuring tank 640 and the inner surface of the channel 620 are smooth surfaces to facilitate the sliding of liquid (blood and reaction solution). In some embodiments, the arithmetical mean deviation of the profile Ra of the smooth surfaces is 0.2-0.4 μm (micrometer).

In some embodiments, the distance between the first main light-penetrating part 642 and the second main light-penetrating part 644 is 10 (1±5%) mm, and the capacity of the measuring tank 640 is 0.2 (1±5%) mL.

In some embodiments, the measuring container 60 is made of poly (methyl methacrylate) (PMMA).

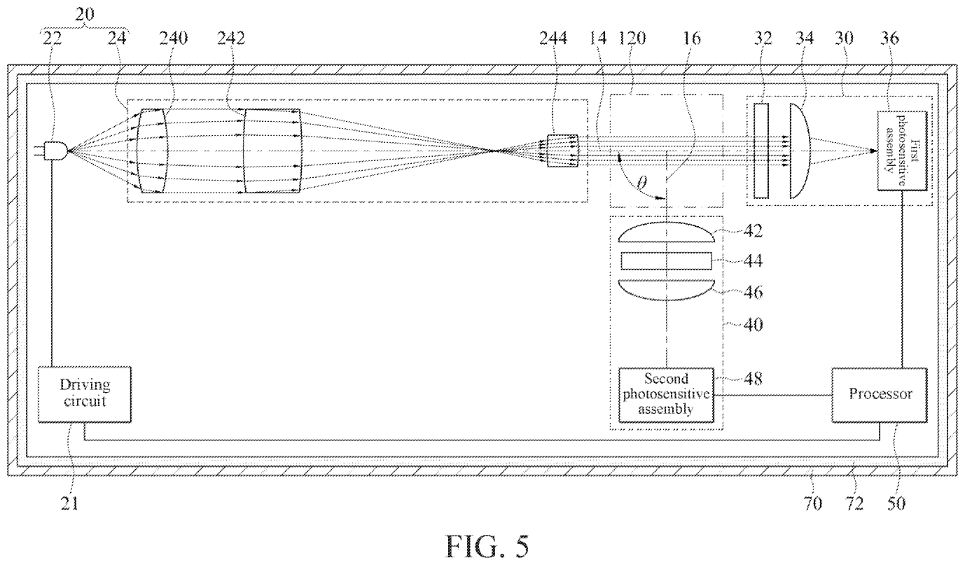

In some embodiments, a manufacturer may provide a hemoglobin advanced glycation end products measuring instrument without the measuring container 60, as shown in FIG. 1 and FIG. 5. This hemoglobin advanced glycation end products measuring instrument includes a measuring base 10, a light emitting unit 20, a first photosensitive assembly 30, a second photosensitive assembly 40 and a processor 50. The measuring base 10 includes a main body 12, a first optical axis 14 and a second optical axis 16. The main body 12 is provided with an accommodated space 120. The second optical axis 16 and the first optical axis 14 are intersected in the accommodated space 120 by an included angle θ. The light emitting unit 20 is positioned at one end of the first optical axis 14 of the measuring base 10, and substantially outputs parallel light beam along the first optical axis 14 when being driven. The parallel light beam at the measuring base 10 (the accommodated space 120) generates second light beam, and first light beam towards the first optical axis 14. The first photosensitive assembly 30 is positioned at the other end of the first optical axis 14 of the measuring base 10, and receives and converts the first light beam with a first preset wavelength into first light intensity. The second photosensitive assembly 40 is positioned at one end of the second optical axis 16 and receives and converts the second light beam with a second preset wavelength into second light intensity. The processor 50 is configured to obtain a hemoglobin advanced glycation end products measuring result according to the first light intensity, the second light intensity, the first standard intensity and the first empty intensity.

In this embodiment, when the measuring container 60 with the to-be-tested liquid is placed in the accommodated space 120, the light emitting unit 20 outputs the parallel light beam with the peak wavelength between 330 and 390 nm when being driven; the second light beam generated by the parallel light beam irradiating the to-be-tested liquid are fluorescence; the first light beam generated are the transmission light beam; the first preset wavelength is between 330 and 390 nm; the second preset wavelength is between 420 and 520 nm; the included angle θ is 45-135 degrees. The rest components of the hemoglobin advanced glycation end products measuring instrument are similar to those mentioned above and will not be described again.

Definitely, there are many other embodiments of the present application, and those skilled in the art can make various corresponding changes and deformations according to the present application without departing from the spirit and essence of the present application, but these corresponding changes and deformations should belong to the protection scope of the claims of the present application.

What is claimed is:

1. A hemoglobin advanced glycation end products measuring instrument, comprising:
a measuring base comprising a main body, a first optical axis and a second optical axis, the main body being provided with an accommodated space, the second optical axis and the first optical axis being intersected in the accommodated space by an included angle;
a measuring container accommodating to-be-tested liquid and being positioned in the accommodated space to enable the to-be-tested liquid to correspond to the intersection of the first optical axis and the second optical axis;
a light emitting unit being positioned at one end of the first optical axis of the measuring base, and outputting parallel light beam along the first optical axis when being driven, the parallel light beam irradiating the to-be-tested liquid to generate fluorescence and a transmission light beam towards the other end of the first optical axis;
a first photosensitive assembly being positioned at the other end of the first optical axis of the measuring base, and receiving and converting the transmission light beam with a first preset wavelength into a first light intensity;
a second photosensitive assembly being positioned at one end of the second optical axis, and receiving and converting the fluorescence with a second preset wavelength into a second light intensity; and
a processor obtaining a hemoglobin advanced glycation end products measuring result according to the first light intensity, the second light intensity, a first standard intensity and a first empty intensity.

2. The hemoglobin advanced glycation end products measuring instrument according to claim 1, wherein the light emitting unit comprises:
a light emitting diode emitting a light source when being driven; and
a first lens group receiving the light source and outputting the parallel light beam.

3. The hemoglobin advanced glycation end products measuring instrument according to claim 2, wherein the first lens group comprises:
a first lens receiving the light source and outputting a convergent light beam;
a second lens receiving the convergent light beam and outputting a focused light beam; and
a third lens receiving the focused light beam and outputting the parallel light beam.

4. The hemoglobin advanced glycation end products measuring instrument according to claim 1, wherein the light emitting unit comprises:
a laser assembly generating laser when being driven; and
a beam reducer group reducing the laser to output the parallel light beam.

5. The hemoglobin advanced glycation end products measuring instrument according to claim 1, wherein the first preset wavelength and the second preset wavelength have no overlap.

6. A measuring container for the hemoglobin advanced glycation end products measuring instrument according to claim 5, comprising:
a liquid inlet section being provided with a channel and an opening communicating with the channel; and
a measuring section being connected to the liquid inlet section and being provided with a measuring tank, the measuring tank communicating with the channel and accommodating to-be-tested liquid, the measuring tank being provided with a first main light-penetrating part, a second main light-penetrating part and a side light-penetrating part, the first main light-penetrating part and the second main light-penetrating part corresponding to the first optical axis, and the side light-penetrating part corresponding to the second optical axis.

7. The hemoglobin advanced glycation end products measuring instrument according to claim 1, wherein an upper limit of the first preset wavelength is 20 nm smaller than a lower limit of the second preset wavelength.

8. A measuring container for the hemoglobin advanced glycation end products measuring instrument according to claim 7, comprising:
a liquid inlet section being provided with a channel and an opening communicating with the channel; and
a measuring section being connected to the liquid inlet section and being provided with a measuring tank, the measuring tank communicating with the channel and accommodating to-be-tested liquid, the measuring tank being provided with a first main light-penetrating part, a second main light-penetrating part and a side light-penetrating part, the first main light-penetrating part and the second main light-penetrating part corresponding to the first optical axis, and the side light-penetrating part corresponding to the second optical axis.

9. The hemoglobin advanced glycation end products measuring instrument according to claim 1, wherein the first preset wavelength is between 330 and 390 nanometers (nm), the second preset wavelength is between 420 and 520 nm.

10. A measuring container for the hemoglobin advanced glycation end products measuring instrument according to claim 9, comprising:
a liquid inlet section being provided with a channel and an opening communicating with the channel; and
a measuring section being connected to the liquid inlet section and being provided with a measuring tank, the measuring tank communicating with the channel and accommodating to-be-tested liquid, the measuring tank being provided with a first main light-penetrating part, a second main light-penetrating part and a side light-penetrating part, the first main light-penetrating part and the second main light-penetrating part corresponding to the first optical axis, and the side light-penetrating part corresponding to the second optical axis.

11. The hemoglobin advanced glycation end products measuring instrument according to claim 1, wherein the included angle is between 45-135 degrees.

12. The hemoglobin advanced glycation end products measuring instrument according to claim 1, wherein the hemoglobin advanced glycation end products measuring result is a measuring value or a glycation rate code.

13. The hemoglobin advanced glycation end products measuring instrument according to claim 1, wherein the processor obtains the hemoglobin advanced glycation end products measuring result according to the following equation:
M₁=M₀*(N₀−N₁)/(N₀−N)
M₁ represents the hemoglobin advanced glycation end products measuring result, M₀ represents the second light intensity, NO represents the first empty intensity, N₁ represents the first standard intensity, and N represents the first light intensity.

14. A measuring container for the hemoglobin advanced glycation end products measuring instrument according to claim 13, comprising:
a liquid inlet section being provided with a channel and an opening communicating with the channel; and
a measuring section being connected to the liquid inlet section and being provided with a measuring tank, the measuring tank communicating with the channel and accommodating to-be-tested liquid, the measuring tank being provided with a first main light-penetrating part, a second main light-penetrating part and a side light-penetrating part, the first main light-penetrating part and the second main light-penetrating part corresponding to the first optical axis, and the side light-penetrating part corresponding to the second optical axis.

15. The hemoglobin advanced glycation end products measuring instrument according to claim 1, wherein the first photosensitive assembly comprises a first optical filter, a fourth lens and a first photosensitive element are sequentially arranged from the measuring container to the other end of the first optical axis; the first optical filter enables the transmission light beam with the wavelength between 330 and 390 nm to transmit through; the fourth lens focuses the transmission light beam transmitting through the first optical filter on the first photosensitive element; and the first photosensitive element receives and converts the transmission light beam focused by the fourth lens into the first light intensity.

16. The hemoglobin advanced glycation end products measuring instrument according to claim 1, wherein the second photosensitive assembly comprises a fifth lens, a second optical filter, a sixth lens and a second photosensitive element are sequentially arranged from the measuring container to one end of the second optical axis; the fifth lens receives the fluorescence and outputs a parallel beam of fluorescence; the second optical filter enables the parallel beam of fluorescence with the wavelength between 420 and 520 nm to transmit through; the sixth lens focuses the parallel beam of fluorescence transmitting through the second optical filter on the second photosensitive element; and the second photosensitive element receives and converts focused the fluorescence into the second light intensity.

17. A measuring container for the hemoglobin advanced glycation end products measuring instrument according to claim 1, comprising:
a liquid inlet section being provided with a channel and an opening communicating with the channel;
a measuring section being connected to the liquid inlet section and being provided with a measuring tank, the measuring tank communicating with the channel and accommodating to-be-tested liquid, the measuring tank being provided with a first main light-penetrating part, a second main light-penetrating part and a side light-penetrating part, the first main light-penetrating part and the second main light-penetrating part corresponding to the first optical axis, and the side light-penetrating part corresponding to the second optical axis.

18. The measuring container according to claim 17, wherein the first main light-penetrating part and the second main light-penetrating part enable the parallel light beam to transmit through; and the side light-penetrating part enables the fluorescence to transmit through.

19. The measuring container according to claim 17, wherein an inner surface of the measuring tank and an inner surface of the channel are smooth surfaces.

20. A hemoglobin advanced glycation end products measuring instrument, comprising:
a measuring base comprising a main body, a first optical axis and a second optical axis, the main body being provided with an accommodated space, and the second optical axis and the first optical axis being intersected in the accommodated space by an included angle;
a light emitting unit being positioned at one end of the first optical axis of the measuring base and outputting parallel light beam along the first optical axis when being driven, the parallel light beam at the measuring base generating second light beam, and first light beam towards the other end of the first optical axis;
a first photosensitive assembly being positioned at the other end of the first optical axis of the measuring base, and receiving and converting the first light beam with a first preset wavelength into first light intensity;
a second photosensitive assembly being positioned at one end of the second optical axis and receiving and converting the second light beam with a second preset wavelength into second light intensity; and
a processor obtaining a hemoglobin advanced glycation end products measuring result according to the first light intensity, the second light intensity, first standard intensity and first empty intensity.
